(19) United States
(12) Patent Application Publication
(10) Pub. No.: US 2024/0148247 A1
(43) Pub. Date: May 9, 2024

(54) SLIT LAMP SYSTEM, MOUNTING BRACKET THEREFORE, AND METHOD OF IMAGING AN EYE OF A PATIENT

(71) Applicant: LIGHTX INNOVATIONS INC., Chambly (CA)

(72) Inventors: Jean-Mathieu GAGNON, Chambly (CA); Sébastien GAGNE, Blainville (CA); Mathieu CHAMPAGNE, Dunham (CA)

(21) Appl. No.: 18/548,388

(22) PCT Filed: Mar. 2, 2022
(86) PCT No.: PCT/CA2022/050292

§ 371 Date: Aug. 30, 2023

Related U.S. Application Data
(60) Provisional application No. 63/155,804, filed on Mar. 3, 2021.

(51) Int. Cl. A61B 3/135; A61B 3/13; A61B 3/14

(52) U.S. Cl. CPC A61B 3/135; A61B 3/132; A61B 3/14

(57) ABSTRACT

There is described a slit lamp system for imaging an eye of a patient. The slit lamp system generally having: a frame; an illumination source assembly mounted to said frame and adapted to illuminate said eye of said patient into one or more illumination patterns; a binocular imaging assembly mounted to said frame and adapted to image said eye of said patient during said illuminating, said imaging including forming two eye imaging paths transversally spaced-apart from one another and leading away from said frame; and a mounting bracket mounted to said binocular imaging assembly and having two transversally spaced-apart camera receivers, each camera receiver being adapted to receive a corresponding camera for simultaneously capturing two images from said two eye imaging paths.

### FIELD

The improvements generally relate to eye examination and more specifically relates to eye examination using binocular-type slit lamps.

### BACKGROUND

A binocular-type slit lamp is an instrument having an illumination source assembly that can shine a thin strip of light into the eye of a patient, and a binocular microscope for observing the illuminated eye through two ocular elements for examination purposes. Slit lamps are generally operated by optometrists, ophthalmologist and other eye care professionals as they typically require a high level of training to suitably illuminate some specific parts of the eye in precise conditions and assess the condition of the illuminated eye. Although existing slit lamps are satisfactory to a certain degree, there remains room for improvement, especially in imaging the illuminated eye.

### SUMMARY

It was found that there is a need in the industry for capturing images of the eye of a patient in a stereoscopic manner using a binocular-type slit lamp, thereby enabling a perception of depth in the resulting stereoscopic image which can help the eye care professionals in the eye examination. However, existing slit lamp systems either provide limiting monoscopic imaging capabilities or stereo-imaging capabilities requiring optical assemblies with beam splitter(s), lens(es), and/or mirror(s) which add to the cost and complexity of the resulting slit lamp systems.

In accordance with a first aspect of the present disclosure, there is provided a slit lamp system for imaging an eye of a patient, said slit lamp system comprising: a frame; an illumination source assembly mounted to said frame and adapted to illuminate said eye of said patient into one or more illumination patterns; a binocular imaging assembly mounted to said frame and adapted to image said eye of said patient during said illuminating, said imaging including forming two eye imaging paths transversally spaced-apart from one another and leading away from said frame; and a mounting bracket mounted to said binocular imaging assembly and having two transversally spaced-apart camera receivers, each camera receiver being adapted to receive a corresponding camera for simultaneously capturing two images from said two eye imaging paths.

In accordance with a second aspect of the present disclosure, there is provided a mounting bracket for use with a slit lamp having a frame and a binocular imaging assembly mounted to said frame, said binocular imaging assembly comprising two ocular elements transversally spaced-apart from one another, the mounting bracket comprising: a body having a first side with two transversally spaced-apart ocular mounting members, a second side opposite said first side having two transversally spaced-apart camera receivers, and two transversally spaced-apart camera apertures extending through said body between said first side and said second side, wherein, during use, said ocular mounting members are removably mounted to said two ocular elements of said binocular imaging assembly, and said two camera receivers removably receive cameras facing said camera apertures and exposed to said ocular elements.

In accordance with a third aspect of the present disclosure, there is provided a method of imaging an eye of a patient using a slit lamp having a binocular imaging assembly, the method comprising: mounting cameras across eye imaging paths of said binocular imaging assembly; upon receiving an input, communicating a synchronization signal to at least one of said cameras; the two cameras simultaneously capturing an image of the eye through a respective one of said ocular elements based on said synchronization signal; and forming a stereoscopic image based on said captured images. It is anticipated that by using two camera-equipped mobile devices, or simply cameras, each capturing a corresponding image from one of the two ocular elements of the binocular-type slit lamp, the amount of captured information can be doubled compared to existing camera-equipped slit lamps. As such, the resolution of the resulting stereoscopic image can be doubled compared to stereo-imaging slit lamps requiring optical assemblies with beam splitter(s), lens(es), and/or mirror(s) discussed above.

Many further features and combinations thereof concerning the present improvements will appear to those skilled in the art following a reading of the instant disclosure.

### DESCRIPTION OF THE FIGURES

In the figures,

FIG. 1 is an oblique view of an example of a slit lamp system, showing a binocular imaging assembly, a mounting bracket, and mobile devices in an exploded manner, in accordance with one or more embodiments;

FIG. 1A is a top view of the slit lamp system of FIG. 1, with the mobile devices being mounted to the binocular imaging assembly using the mounting bracket, in accordance with one or more embodiments;

FIGS. 2A and 2B are exemplary images generated by cameras of the mobile devices of FIG. 1A, in accordance with one or more embodiments;

FIG. 3 is an exemplary stereoscopic image obtained based on the images of FIGS. 2A and 2B, in accordance with one or more embodiments;

FIG. 4A is a rear view of another example of a mounting bracket, showing adjustable mobile device receivers, in accordance with one or more embodiments;

FIG. 4B is a front view of the mounting bracket of FIG. 4A, showing adjustable ocular mounting members, in accordance with one or more embodiments;

FIG. 5 is a schematic view of an example of a computing device of an exemplary controller, in accordance with one or more embodiments;

FIG. 6 is a block diagram of a software application the exemplary controller, in accordance with one or more embodiments;

FIG. 7 is an oblique and exploded view of another example of a slit lamp system, showing a mounting bracket to be mounted between a binocular scope and corresponding ocular elements, in accordance with one or more embodiments;

FIG. 7A is an oblique view of the slit lamp system of FIG. 7, showing the mounting bracket mounted between the binocular scope and the ocular elements, in accordance with one or more embodiments;

FIG. 8 is a front elevation view another example of a slip lamp system, showing a mounting bracket mounting mobile devices directly to a binocular scope, in accordance with one or more embodiments; and

FIG. 8A is a top view of the slit lamp system of FIG. 8, in accordance with one or more embodiments.

### DETAILED DESCRIPTION

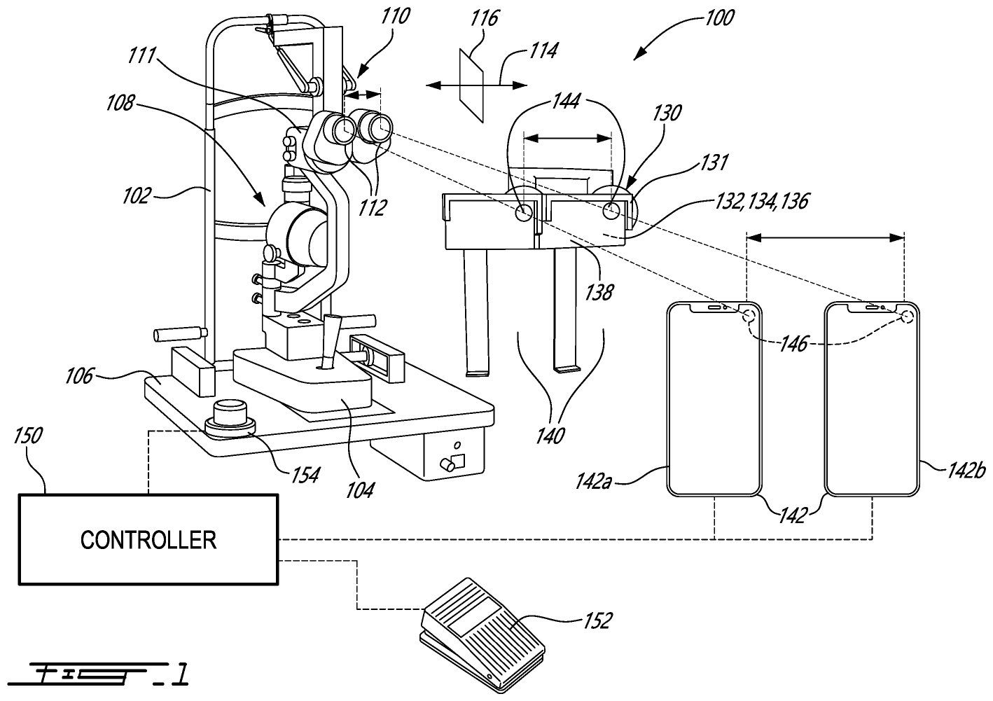

FIG. 1 shows an example of a slit lamp system 100 for imaging an eye of a patient, in accordance with a specific embodiment. As shown, the slit lamp system 100 has a frame 102 supporting components of the slit lamp system 100. The frame 102 generally has a base 104 resting on a support surface 106 such as a table top, a floor and the like.

As depicted, the slit lamp system 100 has an illumination source assembly 108 adapted to illuminate the eye of the patient in one or more illumination patterns. Examples of such illumination patterns can include, but are not limited to, diffuse illumination, direct focal illumination, tangential illumination, retroillumination, indirect illumination, sclerotic scatter illumination, and any combination thereof. The illumination source assembly 108 can be positioned above the head of the patient, such as in Haag Streit type slit lamps, or below the head of the patient, such as in Zeiss type slit lamps. As such, the illumination source assembly 108 can include a variety of other optical components, such as shutter(s), mirror(s), diffuser(s), filter(s) and the like to propagate, carry and/or modify the light generated by a slit illuminator and a background illuminator, for instance.

The slit lamp system 100 also has an imaging assembly 110 which is mounted to the frame 102. The imaging assembly 110 has a number of optical elements adapted to image the eye of the patient during illumination as provided by the illumination source assembly 108. As shown, the imaging assembly 110 is of the binocular type. More specifically, the binocular imaging assembly 110 has a binocular scope 111 optically coupled to two ocular elements 112 which are transversally spaced-apart from one another. In this disclosure, the transverse orientation 114 is generally perpendicular to a sagittal plane 116 of the slit lamp system 100 and to the vertical orientation. As best shown in FIG. 1A, the binocular scope 111 and the two ocular elements 112 collectively form images of the eye E propagating along corresponding eye imaging paths, with each ocular elements forming a corresponding image at an imaging plane 118 axially-spaced apart along an axis A of the ocular element 112 during the imaging process. As shown, the imaging assembly 110 can include a variety of other optical components, such as shutter(s), mirror(s), diffuser(s), filter(s) and the like to propagate, carry and/or modify the light incoming from the eye E of the patient. In conventional eye examination, a health care professional can view the images of the eye E by placing her/his eyes at the imaging planes 118 formed before the ocular elements 112.

Referring back to FIG. 1, the slit lamp system 100 has a mounting bracket 130 to be removably attached to the slit lamp system 100, and more specifically to the ocular elements 112 in this specific embodiment. The mounting bracket 130 has a body 131 with a first face 132 which has features 134 being directly or indirectly mounted to the ocular elements 112. More specifically, in this embodiment, the mounting bracket 130 has ocular mounting members 136 which are attachable, clipable or otherwise mountable on the ocular elements 112 of the binocular imaging assembly 110. In some other embodiments, the mounting bracket 130 may be directly or indirectly mounted with respect to the ocular elements 112. In other words, the mounting bracket 130 may be mounted directly to the frame, but place before the ocular elements 112. The mounting bracket 130 has a second face 138 opposite to the first face 132. As shown, the second face 138 has two transversally spaced-apart camera receivers 140 which are each configured to removably receive a corresponding one of a pair of cameras. As shown, the cameras can be part of corresponding mobile devices 142. As such, in some embodiments, the camera receivers 140 can be provided in the form of mobile device receivers. The mounting bracket 130 also has two transversally spaced-apart camera through apertures 144 extending between the first and second faces 132 and 138 of the mounting bracket 130.

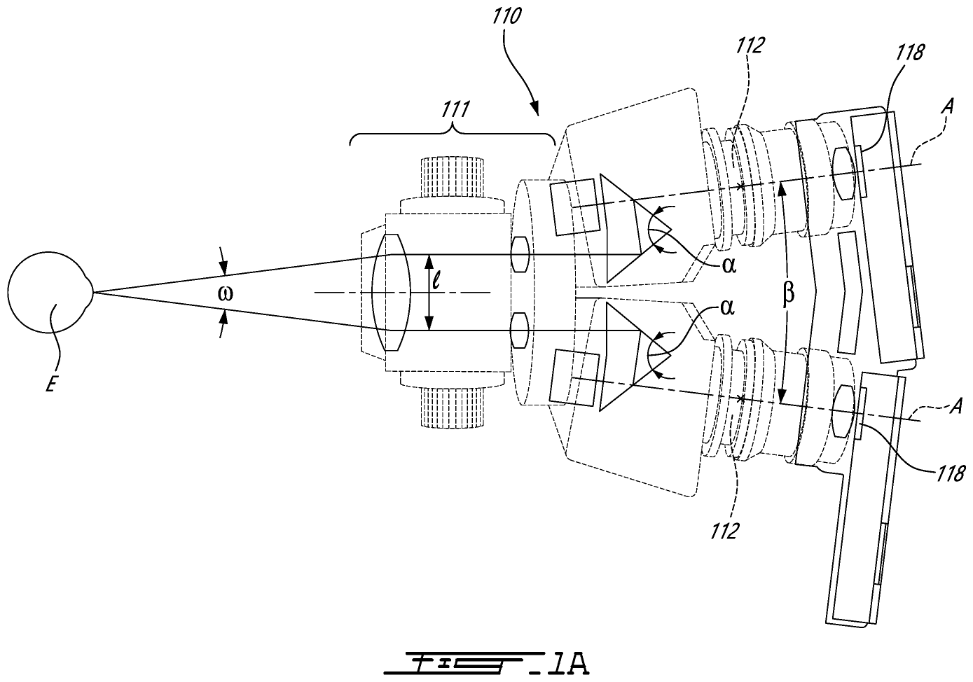

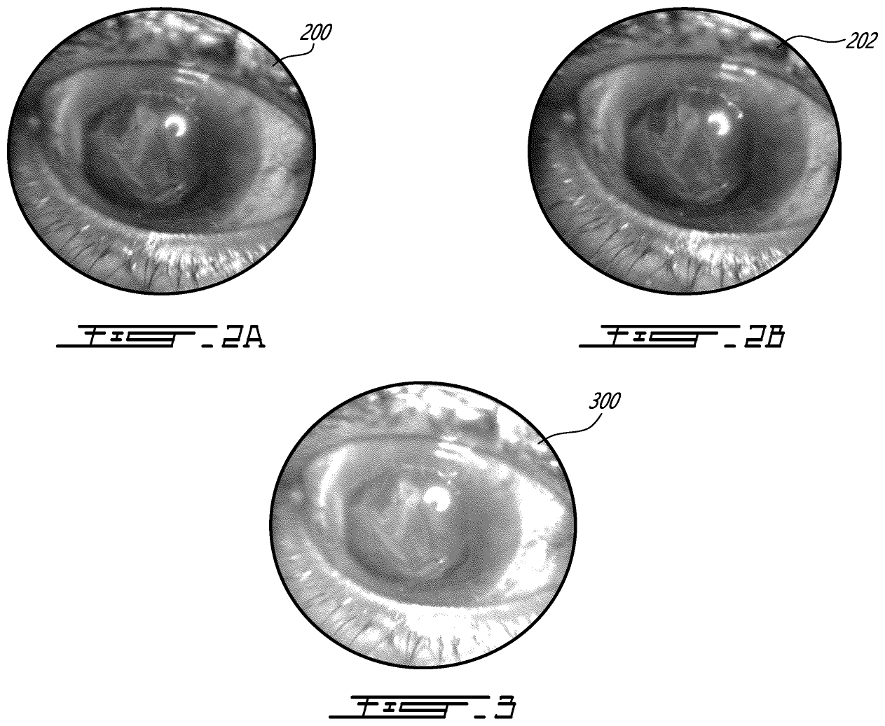

In this embodiment, there are provided two mobile devices 142 each having a respective camera 146. The type of mobile device can include, but is not limited to, a smartphone such as the iPhone® (any generation), the Android® phone (any generation) and the like, an electronic tablet such as the iPad® (any generation, the Android® tablet (any generation) and the like. The mobile devices can be of a similar type in some embodiments, or of dissimilar types in some other embodiments. In some embodiments, the camera 146 needs not to be provided as part of a mobile device. For instance, the cameras may be standalone cameras including, but not limited to, point and shoot cameras, digital single lens reflex (DSLR) cameras, and the like. The cameras 146 typically have a resolution greater than 2 megapixels, preferably greater than 4 megapixels, and more preferably more than 8 megapixels. When the two mobile devices 142 are suitably received in the camera receivers 140 of the mounting bracket 130, the cameras 146 of the two mobile devices 142 are exposed to the imaging planes of the binocular imaging assembly 110 via the camera apertures 144. As such, the mobile devices 142 can simultaneously capture the images of the eye of the patient formed at the imaging planes of the two ocular elements 112, examples of which are shown in FIGS. 2A and 2B. More specifically, FIG. 2A shows a first image 200 captured by a first one of the two mobile devices whereas FIG. 2B shows a second image 202 captured by a second one of the two mobile devices. By associating the two simultaneously captured images to one another, a stereo image pair can be obtained thereby allowing three-dimensional perception of the eye of the patient. The stereo image pair can be processed to obtain a stereoscopic image 300, an example of which is shown in FIG. 3.

Still referring to FIG. 1, the slit lamp system 100 has a controller 150 having a processor and a memory having instructions stored thereon that when executed by the processor perform at least some steps. For instance, in some embodiments, the controller performs a step of receiving an input, and upon reception of that input, another step of generating a synchronization signal triggering the cameras 146 of the mobile devices 142 to simultaneously capture the images formed by the ocular elements 112. The controller 150 can be communicatively coupled to either one or both of the mobile devices 142. The controller 150 can also be communicatively coupled to input sources such as a foot pedal 152, a finger button 154, or any other suitable input sources accessible by a health care professional operating the slit lamp system 100. The communication between the controller 150 and these components can be wired, wireless or a combination of both, depending on the embodiment. For instance, in some embodiments, upon activation of any one of the input sources 152 and 154, the controller 150 can send a synchronization signal to either one or both of the mobile devices 142 so that they synchronize each other in the image capture process. In some embodiments, the two mobile devices 142 operate independently and each captures an image upon reception of the synchronization signal generated by the controller 150. In some other embodiments, one of the two mobile devices 142 is a primary mobile device 142a and the other one of the two mobile devices 142 is a secondary mobile device 142b listening for instructions incoming from the primary mobile device 142a. In these embodiments, the synchronization signal may be communicated only to the primary mobile device 142a which can thereafter coordinate itself with the secondary mobile device 142b for image capture synchronization. In some embodiments, such as the one illustrated in FIG. 1, the controller 150 can be an entity separate from the mobile devices 142 and in communication therewith. However, in some other embodiments, it is envisaged that the controller 150 may be part of either one of both of the mobile devices 142, or part of the mounting bracket 130.

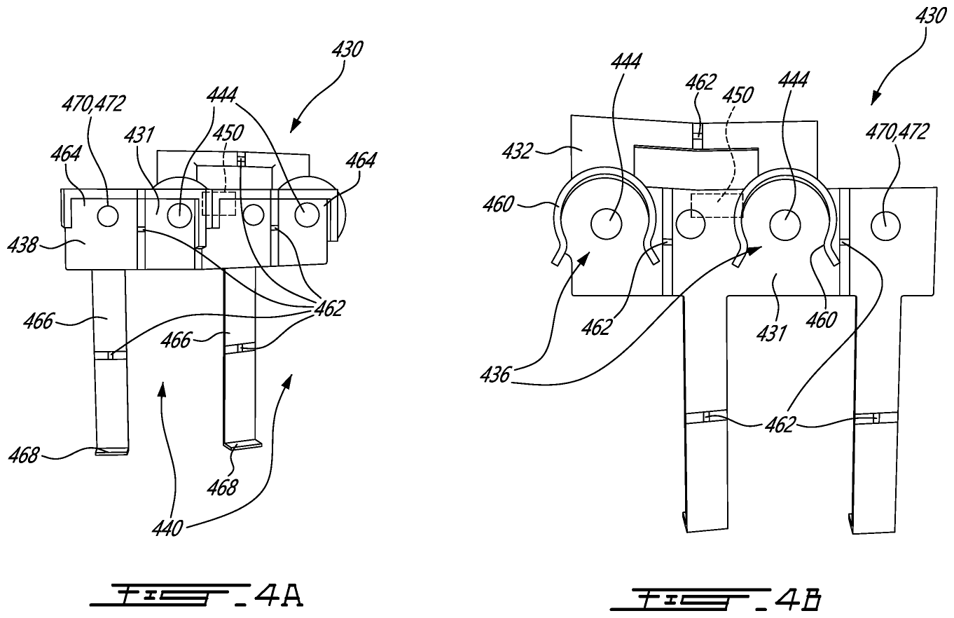

FIGS. 4A and 4B shows rear and front views of another example of a mounting bracket 430. As shown, the mounting bracket 430 has a body 431 having a first side 432 with two transversally spaced-apart ocular mounting members 436, and a second side 438 opposite the first side 432 with two transversally spaced-apart camera receivers 440. As shown, two transversally spaced-apart camera apertures 444 extend through the body 431 between the first side 432 and the second side 438. As discussed with reference to FIG. 1, during use, the ocular mounting members 436 are removably mounted to ocular elements of the corresponding binocular-type slit lamp, whereas two mobile devices are to be removably mounted to the camera receivers 440 in a way that expose cameras thereof before the camera apertures 444.

As shown, the ocular mounting members 436 are provided in the form of partially annular members 460 protruding away from the second side 438. The partially annular members 460 can be snugly received around the circular perimeter of the ocular elements of the binocular-type slit lamp. In this embodiment, the ocular mounting members 436 are transversally adjustable with respect to one another to move them closer or apart from one another as may deemed necessary to fit different ocular elements. This type of adjustability may be provided by mechanically spacing body portions closer or farther from one another as can be allowed by adjustment mechanisms 462. In some embodiments, the ocular mounting members 436 can be adjustable to be mountable to ocular elements of different diameters. The mounting bracket 430 may be adjustable to pivot one of the ocular mounting members 436 towards or away from the other one of the ocular mounting members, to fit on ocular elements forming different angles relative to one another.

In this embodiment, each of the camera receivers 440 is sized and shaped so as to snappingly receive a typical mobile device. For instance, the camera receivers 440 can have an upper perimeter portion 464 protruding away from the first side 432 and which can snugly receive a corresponding upper perimeter portion of the mobile device. An arm 466 having a bottom clipping member 468 can extends vertically downwards from the upper perimeter portion to receive a corresponding bottom portion of the mobile. Once secured in the camera receiver, the mobile device can be removed therefrom by pulling a tab of the bottom clipping member 468 which will free the bottom portion of the mobile device from the camera receiver and in turn free the whole mobile device. In this embodiment, the camera receivers 440 are transversally and vertically adjustable with respect to one another to move them closer or apart from one another as may deemed necessary to fit different mobile devices. This type of adjustability may be provided by mechanically spacing body portions closer or farther from one another as can be allowed by the adjustment mechanisms 462.

In some specific embodiments, the position of the camera apertures 464 can be moved thanks to an aperture moving mechanism (not shown). In some other embodiments, the body 431 is provided with a plurality of camera aperture pairs 470, and a plurality of camera aperture plugs 472 which can be snugly fit into at least some of the camera aperture pairs 470. Depending on the type of mobile device to be received in the camera receivers, some of the camera aperture pairs can be blocked by the camera aperture plugs 474 and other, exposed. In this way, the mounting bracket can be adjusted to receive different types of mobile devices.

It may be envisaged that the mounting bracket may have a controller 450, such as the controller 150 described above with respect to FIG. 1, embedded in the body 431 thereof. In these embodiments, the controller can receive an input, and, upon the reception of the input, generate a synchronization signal to trigger the cameras of the mobile devices received therein for simultaneous image capture purposes. The synchronization signal may be communicated to both mobile device or to a primary one of the mobile devices, depending on the embodiment.

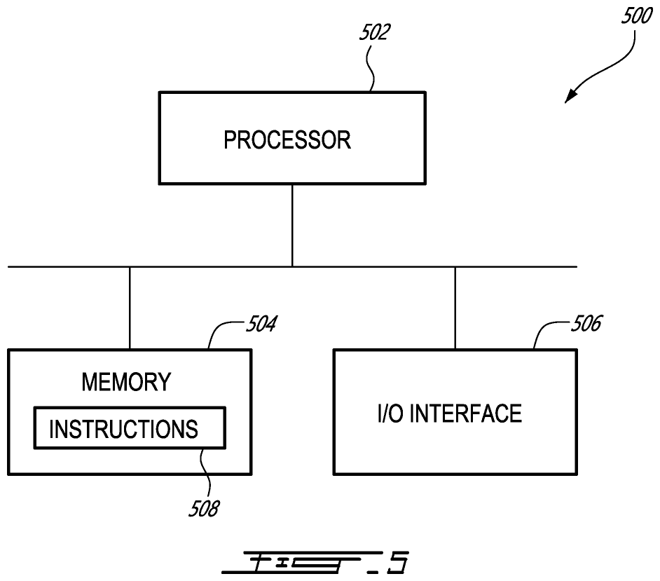

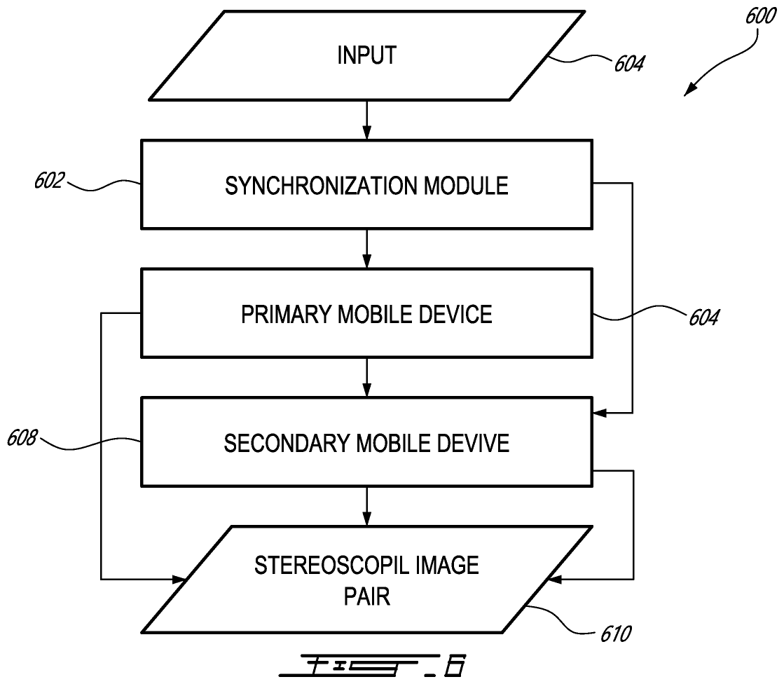

The controllers 150 and 450 can be provided as a combination of hardware and software components. The hardware components can be implemented in the form of a computing device 500, an example of which is described with reference to FIG. 5. Moreover, the software components of the controllers 150 and 450 can be implemented in the form of a software application 600, an example of which is described with reference to FIG. 6.

Referring to FIG. 5, the computing device 500 can have a processor 502, a memory 504, and I/O interface 506. Instructions 508 for generating a synchronization signal and for simultaneously capturing images can be stored on the memory 504 and accessible by the processor 502.

The processor 502 can be, for example, a general-purpose microprocessor or microcontroller, a digital signal processing (DSP) processor, an integrated circuit, a field programmable gate array (FPGA), a reconfigurable processor, a programmable read-only memory (PROM), or any combination thereof.

The memory 504 can include a suitable combination of any type of computer-readable memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

Each I/O interface 506 enables the computing device 500 to interconnect with one or more input devices, such as an input source such as a foot pedal or a finger button, or with one or more output devices such as a monitor, an external memory and/or a remote network.

Each I/O interface 506 enables the controllers 150 and 450 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

Referring now to FIG. 6, an example of a software application 600 is shown. In this specific embodiment, the software application 600 has a synchronization module 602 which is communicatively coupled to an input source 604 and to at least one of the mobile devices 606 and 608. In some embodiments, the software application 600 is stored on the memory 504 and accessible by the processor 502 of the computing device 500. More specifically, upon receiving an input 602, the synchronization module 602 can generate a synchronization signal to be transmitted to either one of both the mobile devices 606 and 608 for simultaneous image capture to result in a stereoscopic image 610 after processing of the captured images. The computing device 500 and the software application 600 described above are meant to be examples only. Other suitable embodiments of the controller 150 can also be provided, as it will be apparent to the skilled reader.

In some aspect, there is described a method of imaging an eye of a patient using a slit lamp having a binocular imaging assembly with two ocular elements transversally spaced-apart from one another. In this aspect, the method has a number of steps to be performed.
At a first step, cameras of two mobile devices are each mounted to a respective one of the two ocular elements of the binocular imaging assembly.
At a second step, upon receiving an input, a synchronization signal is communicated to at least one of the mobile devices.
In a third step, the two mobile device cameras simultaneously capture an image of the eye through a respective one of the ocular elements based on the synchronization signal.

As described above, the synchronization signal can be communicated to both the mobile devices simultaneously in some embodiments. In some other embodiments, the synchronization signal can be communicated to a primary one of the mobile devices, with the primary mobile device then coordinating with a secondary mobile device for the simultaneous capture.

In some embodiments, the mobile devices are removably mounted to the two ocular elements using a mounting bracket mounted to the two ocular elements. The mounting bracket can be in turn removably mounted to the two ocular elements. It is envisaged that the mounting bracket can be adjustable to fit different types of ocular elements and/or different types of mobile devices.

Although the embodiments described above present a mounting bracket mounting the cameras, or corresponding mobile devices, directly to the ocular elements, some other embodiments may differ. For instance, the mounting bracket may be used to mount the cameras anywhere else along the eye imaging paths of the binocular imaging assembly. In some embodiments, the mounting bracket may include one or more beam splitters and/or reflective surfaces deviating partially or wholly light propagated along the eye imaging paths.

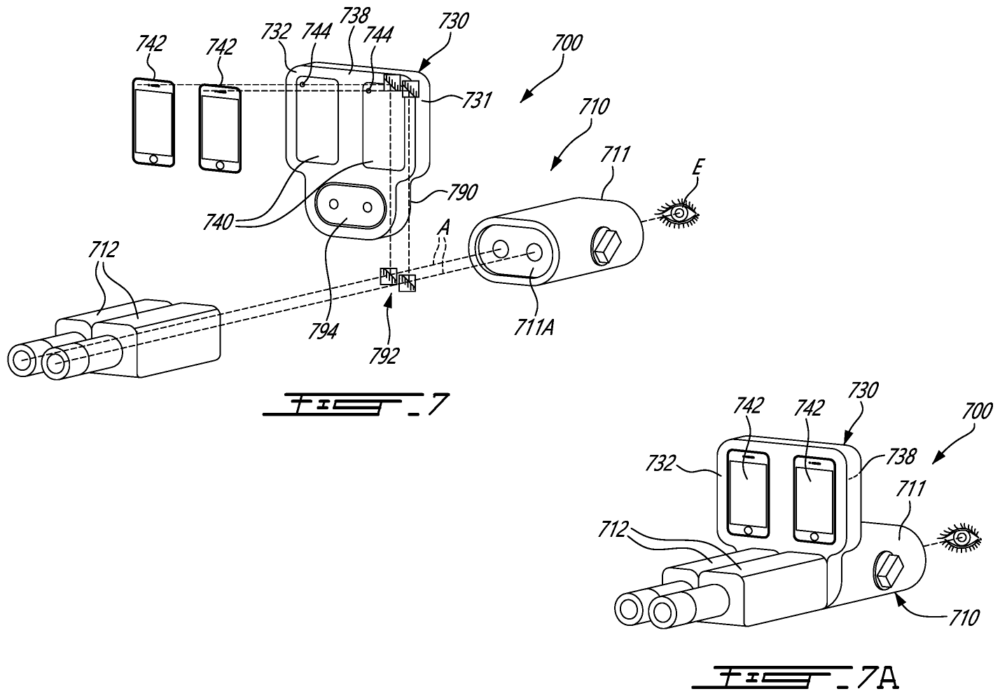

FIG. 7 shows an example of a slit lamp system 700 for imaging an eye E of a patient. As shown, the slit lamp system 700 has a frame, an illumination source, a binocular imaging assembly 710 and a mounting bracket 730 receiving camera-bearing mobile devices 742. In this specific example, the binocular imaging assembly 710 has a binocular scope 711 defining eye imaging paths A running alongside each other and along which images of the eyes E are propagated. The binocular imaging assembly 710 also has ocular elements 712 which are transversally spaced-apart from one another. As can be understood, in the slip lamp system describe with reference to FIG. 1, the ocular elements are directly mounted to the binocular scope for receiving corresponding ones of the eye imaging paths. In this embodiment, the mounting bracket 730 is mounted between the binocular scope 711 and the ocular elements 712. Accordingly, the ocular elements 712 are indirectly mounted to the binocular scope 711 via the mounting bracket 730.

The mounting bracket 730 has a housing 731 having a first side 732 opposite a second side 738, camera receivers 740 side by side from each other on the first side 732, and camera apertures 744 extending through at least the first side 732. The housing 731 of the mounting bracket 730 is also equipped with at least a scope connector 790 connectable to a distal end 711A of the binocular scope 711. As shown, a beam splitter assembly 792 mounted inside the housing 731 of the mounting bracket 730 receives from the binocular scope 711 the images propagated along the eye imaging paths A, and redirect them towards the camera apertures 744. In some embodiments, such as the one shown in FIG. 7, the housing 731 has ocular connectors 794 connectable to the ocular elements 712. As such, the beam splitter assembly 792 allows the redirection of a portion of the light propagated along the eye imaging paths A in a manner which leaves another portion of that light to be propagated along the remainder of the eye imaging paths A to the ocular elements 712. FIG. 7A shows the slit lamp system 700 in a mounted state, with the ocular elements 712 being indirectly mounted to the binocular scope 711 via the mounting bracket 730. As such, with the slit lamp system 700, an health care professional may observe the eye E of the patient via the ocular elements while simultaneously capturing stereoscopic images thereof using the cameras of the two mobile devices.

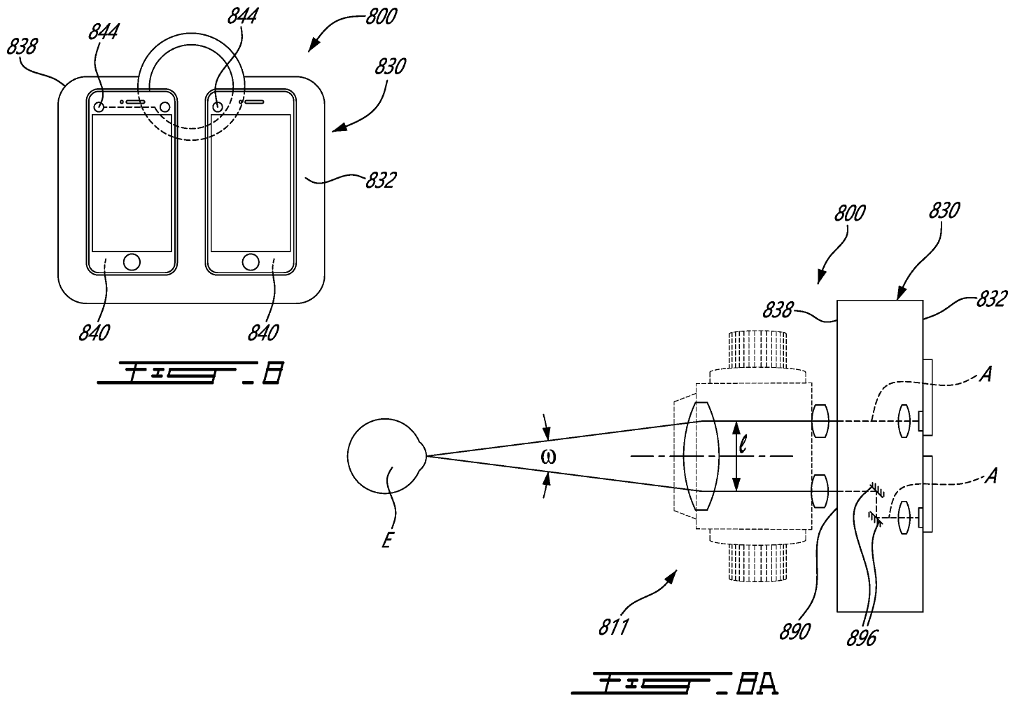

FIG. 8 shows an example of a slit lamp system 800 where the ocular elements are omitted. As shown, the mounting bracket 830 has a first side 832, a second side 838 opposite the first side 832, camera receivers 840 on the first side 832, camera apertures 844 extending from the first side 832 to the second side 838, and a scope connector 890 on the second side 838. As such, the cameras can be positioned across the eye imaging paths A defined by the binocular scope 811 in lieu of the ocular elements. In this embodiment, the mounting bracket 830 can have one or more reflective surfaces 896 redirecting either one or both of the eye imaging paths A towards the camera apertures 844 thereby allowing the simultaneous capture of the images propagated along the eye imaging paths A, as best shown in FIG. 8A.

As can be understood, the examples described above and illustrated are intended to be exemplary only. For instance, the controller can be part of any one or both of the mobile devices or cameras. The scope is indicated by the appended claims.

1. A slit lamp system for imaging an eye of a patient, said slit lamp system comprising:
a frame;
an illumination source assembly mounted to said frame and adapted to illuminate said eye of said patient into one or more illumination patterns;
a binocular imaging assembly mounted to said frame and adapted to image said eye of said patient during said illuminating, said imaging including forming two eye imaging paths transversally spaced-apart from one another and leading away from said frame; and
a mounting bracket mounted to said binocular imaging assembly and having two transversally spaced-apart camera receivers, each camera receiver being adapted to receive a corresponding camera for simultaneously capturing two images from said two eye imaging paths.

2. The slit lamp system of claim 1 wherein said binocular imaging assembly has two ocular elements forming said images at imaging planes along said two eye imaging paths, said mounting bracket being directly or indirectly mounted to said two ocular elements.

3. The slit lamp system of claim 2 wherein said mounting bracket has a first side mounted to said two ocular elements, a second side opposite said first side having said two transversally spaced-apart camera receivers, and two transversally spaced-apart camera apertures extending through between said first side and second side, wherein each camera receiver is adapted to receive a corresponding camera facing a respective one of said camera apertures.

4. The slit lamp system of claim 3 wherein said first side of said mounting bracket is removably mounted to at least one of said two ocular elements.

5. The slit lamp system of claim 3 wherein said cameras are part of corresponding mobile devices, said slit lamp system further comprising two mobile devices received in of said camera receivers, with said cameras facing said camera apertures.

6. The slit lamp system of claim 5 wherein said mobile devices are removably received in said camera receivers.

7. The slit lamp system of claim 1 further comprising a controller communicatively coupled to at least one of said cameras, said controller having a processor and a memory having instructions that when executed by said processor perform the steps of: receiving an input; and, upon said receiving, generating a synchronization signal triggering directly or indirectly said cameras to simultaneously capture said images.

8. The slit lamp system of claim 7 further comprising an input source activatable to generate said input.

9. The slit lamp system of claim 7 wherein said synchronization signal is communicated to both said cameras simultaneously.

10. The slit lamp system of claim 7 wherein said synchronization signal is communicated to a primary one of said cameras, said primary one of said cameras coordinating with a secondary one of said cameras to perform said simultaneous capture.

11. A mounting bracket for use with a slit lamp having a frame and a binocular imaging assembly mounted to said frame, said binocular imaging assembly comprising two ocular elements transversally spaced-apart from one another, the mounting bracket comprising: a body having a first side with two transversally spaced-apart ocular mounting members, a second side opposite said first side having two transversally spaced-apart camera receivers, and two transversally spaced-apart camera apertures extending through said body between said first side and said second side, wherein, during use, said ocular mounting members are removably mounted to said two ocular elements of said binocular imaging assembly, and said two camera receivers removably receive cameras facing said camera apertures and exposed to said ocular elements.

12. The mounting bracket of claim 11 wherein at least one of said ocular mounting members is transversally adjustable to move said ocular mounting members closer or apart from one another.

13. The mounting bracket of claim 11 wherein at least one of said camera receivers is transversally adjustable to move the camera receivers closer or apart from one another.

14. The mounting bracket of claim 11 wherein said body has a plurality of camera apertures pairs, and a plurality of camera aperture plugs snugly received in at least some of said camera apertures of said pairs.

15. The mounting bracket of claim 13 further comprising a controller having a processor and a memory having instructions that when executed by said processor perform the steps of: receiving an input; and, upon said receiving, generating a synchronization signal triggering directly or indirectly said cameras to simultaneously capture said images.

16. The mounting bracket of claim 15 wherein said synchronization signal is communicated to both said cameras.

17. The mounting bracket of claim 15 wherein said synchronization signal is communicated to a primary one of said cameras, said primary one of said cameras coordinating with a secondary one of said cameras to perform said simultaneous capture.

18. A method of imaging an eye of a patient using a slit lamp having a binocular imaging assembly, the method comprising:
mounting cameras across eye imaging paths of said binocular imaging assembly;
upon receiving an input, communicating a synchronization signal to at least one of said cameras;
the two cameras simultaneously capturing an image of the eye through a respective one of said ocular elements based on said synchronization signal; and
forming a stereoscopic image based on said captured images.

19. The method of claim 18 wherein said synchronization signal is communicated to both said cameras simultaneously.

20. The method of claim 18 wherein said synchronization signal is communicated to a primary one of said cameras, said primary one of said cameras coordinating with a secondary one of said cameras for said simultaneous capture.

21-22. (canceled)
